(19) United States
(12) Patent Application Publication
(10) Pub. No.: US 2026/0198929 A1
(43) Pub. Date: Jul. 16, 2026

(54) DEVICES FOR TREATMENT OF VASCULAR DEFECTS

(71) Applicant: MICROVENTION, INC., Aliso Viejo, CA (US)

(72) Inventors: Rangwala HUSSAIN, Villa Park, CA (US); Ronak DHOLAKIA, Aliso Viejo, CA (US)

(21) Appl. No.: 19/127,800

(22) PCT Filed: Nov. 7, 2023
(86) PCT No.: PCT/US2023/036943

§ 371 Date: May 7, 2025

Related U.S. Application Data
(60) Provisional application No. 63/423,941, filed on Nov. 9, 2022.

(51) Int. Cl. A61B 17/12; A61B 90/00

(52) U.S. Cl. CPC A61B 17/12113; A61B 17/12172; A61B 2017/1205; A61B 2090/3966

(57) ABSTRACT

Devices for treatment of aneurysms and methods of delivery are described that include a permeable shell having an open distal end, an inner compressible mesh structure having a lumen, and an outer constraint surrounding at least a portion of the inner compressible structure. The outer constraint may have a variable stiffness. The expanded configuration of permeable shell may be shaped in a torus with the inner compressible mesh structure located along a longitudinal axis of the permeable shell. The outer constraint may be a coil or a hypotube. The outer constraint may have a proximal portion that is stiffer than a distal portion. The stiffer proximal portion of the outer constraint may dampen hemodynamic forces at the proximal end of the permeable shell.

### CROSS-REFERENCE TO RELATED APPLICATIONS

This application is a national stage patent application under 35 U.S.C. § 371 of International Application No. PCT/US23/36943, filed Nov. 7, 2023, which claims the benefit of U.S. Provisional Application No. 63/423,941, filed Nov. 9, 2022, both of which are hereby expressly incorporated by reference in their entireties for all purposes.

### STATEMENT REGARDING FEDERALLY SPONSORED RESEARCH OR DEVELOPMENT

Not applicable.

### FIELD OF THE INVENTION

Embodiments of devices and methods herein are directed to implants for treating aneurysms.

### BACKGROUND OF THE INVENTION

The mammalian circulatory system is comprised of a heart, which acts as a pump, and a system of blood vessels which transport the blood to various points in the body. Due to the force exerted by the flowing blood on the blood vessel the blood vessels may develop a variety of vascular defects. One common vascular defect known as an aneurysm is a result of the abnormal widening of the blood vessel. Typically, vascular aneurysms are formed as a result of the weakening of the wall of a blood vessel and subsequent ballooning and expansion of the vessel wall. If, for example, an aneurysm is present within an artery of the brain, and the aneurysm should burst with resulting cranial hemorrhaging, death could occur.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises. Once access to the aneurysm is gained, the surgeon places a clip across the neck of the aneurysm thereby preventing arterial blood from entering the aneurysm. Upon correct placement of the clip the aneurysm will be obliterated in a matter of minutes. Surgical techniques may be effective treatment for many aneurysms. Unfortunately, surgical techniques for treating these types of conditions include major invasive surgical procedures which often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. One approach to treating aneurysms without the need for invasive surgery involves the placement of sleeves or stents into the vessel and across the region where the aneurysm occurs. Such flow diverter devices maintain blood flow through the vessel while reducing blood pressure applied to the interior of the aneurysm. Certain types of stents are expanded to the proper size by inflating a balloon catheter, referred to as balloon expandable stents, while other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for stents to fit through the microcatheters used in small cerebral blood vessels, their density is usually reduced such that when expanded there is only a small amount of stent structure bridging the aneurysm neck. Thus, they do not block enough flow to cause clotting of the blood in the aneurysm and are thus generally used in combination with vaso-occlusive devices, such as the coils discussed above, to achieve aneurysm occlusion.

Some procedures involve the delivery of embolic or filling materials into an aneurysm. The delivery of such vaso-occlusion devices or materials may be used to promote hemostasis or fill an aneurysm cavity entirely. Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms but suffer from several limitations including poor packing density, compaction due to hydrodynamic pressure from blood flow, poor stability in wide-necked aneurysms, and complexity and difficulty in the deployment thereof as most aneurysm treatments with this approach require the deployment of multiple coils. Coiling is less effective at treating certain physiological conditions, such as wide neck cavities (e.g., wide neck aneurysms) because there is a greater risk of the coils migrating out of the treatment site.

A number of aneurysm neck bridging devices with defect spanning portions or regions have been attempted, however, none of these devices have had a significant measure of clinical success or usage. A major limitation in their adoption and clinical usefulness is the inability to position the defect spanning portion to assure coverage of the neck. Existing stent delivery systems that are neurovascular compatible (i.e., deliverable through a microcatheter and highly flexible) do not have the necessary rotational positioning capability. Another limitation of many aneurysm bridging devices described in the prior art is the poor flexibility. Cerebral blood vessels are tortuous, and a high degree of flexibility is required for effective delivery to most aneurysm locations in the brain.

What has been needed are devices and methods for delivery and use in small and tortuous blood vessels that can substantially block the flow of blood into an aneurysm, such as a cerebral aneurysm, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction, or dislocation.

Intrasaccular occlusive devices are part of a newer type of occlusion device used to treat various intravascular conditions including aneurysms. They are often more effective at treating these wide neck conditions, or larger treatment areas. The intrasaccular devices comprise a structure that sits within the aneurysm and provides an occlusive effect at the neck of the aneurysm to help limit blood flow into the aneurysm. The rest of the device comprises a relatively conformable structure that sits within the aneurysm helping to occlude all or a portion of the aneurysm. Intrasaccular devices typically conform to the shape of the treatment site. These devices also occlude the cross section of the neck of the treatment site/aneurysm, thereby promoting clotting and causing thrombosis and closing of the aneurysm over time. In larger aneurysms, there is a risk of compaction where the intrasaccular device can migrate into the aneurysm and leave the neck region.

Intrasaccular flow diversion devices may be used to treat wide-necked bifurcation aneurysms. A wide-necked bifurcation aneurysm is characterized by parent vessel with two daughter or branch vessels, with the aneurysm located at the bifurcation. These bifurcation aneurysms are observed at internal carotid artery bifurcation, middle cerebral artery bifurcation, anterior cerebral artery bifurcation, and basilar artery bifurcation. Intracranial vascular bifurcations are characterized by impingement of the fluid dynamic forces at the bifurcation junction, which is subsequently distributed among the two bifurcating daughter branch vessels. Intrasaccular flow diversion device(s) implanted in the bifurcation aneurysms experience impingement due to hemodynamic forces at the proximal end. As a result of fluid dynamic impingement, the proximal end may experience compression.

Thus, there is a need for occlusive devices with mechanisms to mitigate the impinging hemodynamic forces.

The following embodiments address this issue by utilizing a device having a preset expanded shape that can conform to fit into and substantially fill numerous sizes of aneurysms.

### SUMMARY OF THE INVENTION

An occlusion device is described that is used to treat a variety of conditions, including aneurysms and neurovascular aneurysms. In some embodiments, the occlusion device is configured as an intrasaccular device.

In many embodiments, the device for treatment of a patient's aneurysm a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and an outer constraint surrounding at least a portion of the inner compressible mesh structure, the outer constraint comprising a variable stiffness along a length of the outer constraint.

In some embodiments, the outer constraint may be a coil.

In some embodiments, the outer constraint comprises a proximal portion and a distal portion, wherein the proximal portion of the outer constraint is stiffer than the distal portion of the outer constraint.

In some embodiments, the outer constraint comprises a coil formed from a wire, wherein the coil comprises a proximal portion having a first pitch and a distal portion having a second pitch, wherein the first pitch is tighter than the second pitch.

In some embodiments, the proximal portion of the coil has a first spring constant and the distal portion has a second spring constant, wherein the first spring constant is lower than the second spring constant.

In other embodiments, the outer constraint may be a hypotube with a plurality of openings.

In many embodiments, a method for treating an aneurysm having an interior cavity and a neck, comprising the steps of: advancing an implant in a microcatheter to a region of interest in an artery, wherein the implant comprises: a permeable shell comprising a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure; and an outer constraint surrounding at least a portion of the inner compressible mesh structure, the outer constraint comprising a variable stiffness along a length of the outer constraint, and a hub at the proximal end of the permeable shell, wherein the first and second ends of each of the plurality of filaments are held in the marker band; deploying the implant within the cerebral aneurysm, wherein the permeable shell expands to the expanded state in the interior cavity of the aneurysm; and withdrawing the microcatheter from the region of interest after deploying the implant.

In many embodiments, a device for treatment of a patient's aneurysm includes a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and a constraining means for located around at least a portion of the inner compressible mesh structure for dampening hemodynamic forces at the proximal end of the permeable shell.

### BRIEF DESCRIPTION OF THE DRAWINGS

These and other aspects, features, and advantages of which embodiments of the invention are capable of will be apparent and elucidated from the following description of embodiments of the present invention, reference being made to the accompanying drawings, in which:

The various figures included show the occlusive device according to one or more embodiments.

FIG. 1A shows an exemplary device for treatment of an aneurysm that includes a stiffening element.

FIG. 1B shows a top view of the device of FIG. 1A.

FIG. 2 shows an exemplary tubular mesh.

FIG. 3A shows a heat-set tubular mesh with a portion having a smaller diameter.

FIG. 3B shows the heat-set tubular mesh of FIG. 3A with a stiffening element.

FIG. 3C shows the heat-set tubular mesh of FIG. 3B after inversion.

FIG. 4 shows a device for treatment of an aneurysm that includes an alternative stiffening element.

FIGS. 5A-5B show exemplary devices deployed within a vascular defect.

### DESCRIPTION OF EMBODIMENTS

The presented embodiments shall generally relate to occlusive devices that can be used to treat aneurysms.

Intrasaccular occlusive devices that include a permeable shell formed from a woven or braided mesh have been described in US 2016/0249935, US 2017/0095254, US 2016/0249934, US 2016/0367260, US 2016/0249937, US 2018/0000489, and US 2019/0223881 all of which are hereby expressly incorporated by reference in their entirety for all purposes.

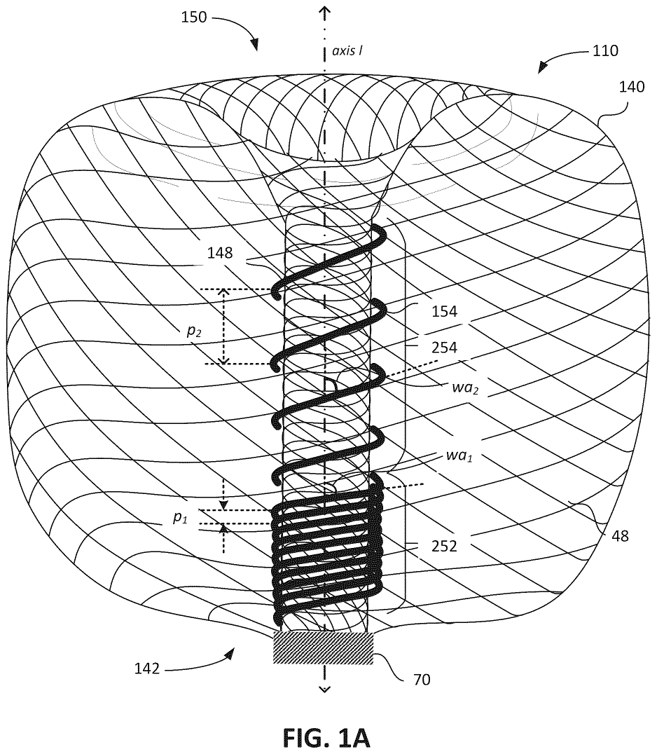

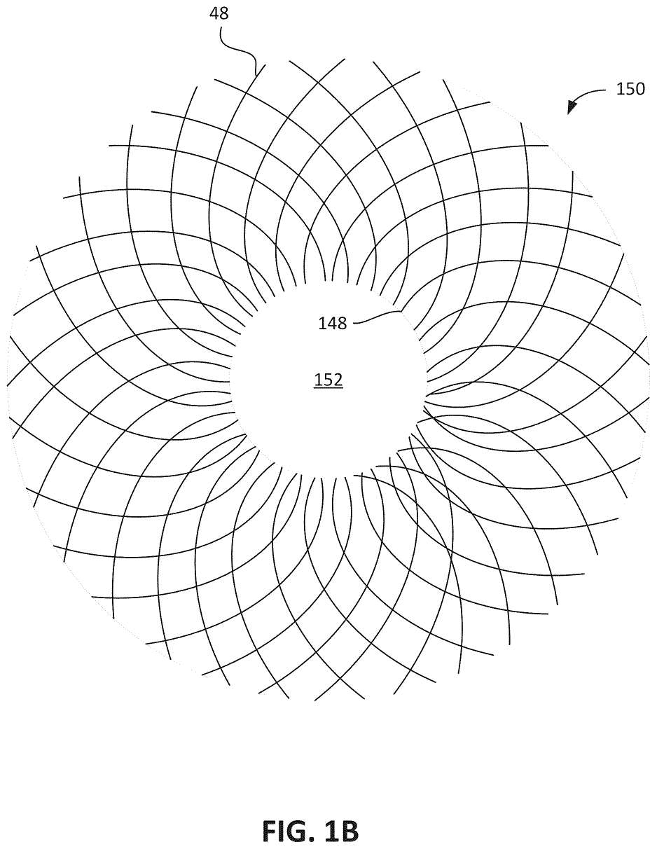

FIGS. 1A-1B depict an intrasaccular device 110 that includes a permeable shell 140 that has an inner compressible structure 148 having a lumen. The inner compressible structure 148 may also be called an inner compressible portion, an inner tubular member, or an inner permeable mesh member. The permeable shell 140 has a proximal end 142, a distal end 150, a longitudinal axis, and may be made from a plurality of elongate resilient filaments 48. As seen in FIG. 1B, the permeable shell may have an open distal end 150 having an opening 152 that communicates with the lumen of the inner compressible structure 148. The filaments may have a woven structure and are secured relative to each other at the proximal end of the permeable shell 140 in a proximal hub 70. Each of the filaments 48 of the plurality of filaments may start at the proximal end 142 of the permeable shell 140, extend to the distal end 150, and extend back to the proximal end 142. Both of the free ends of each of the filaments may be held in a proximal hub 70. In some embodiments, the proximal hub 70 may be radiopaque. In some embodiments, the proximal hub 70 may be surrounded by a marker band. The permeable shell 140 of the device 110 has a radially constrained elongated state configured for delivery within a microcatheter, with the thin woven filaments extending longitudinally from the proximal end to the distal end of each permeable shell radially adjacent each other along a length of the filaments. The permeable shell 140 has an expanded relaxed state with a longitudinally shortened configuration relative to the radially constrained state. In the expanded state, the woven filaments form the self-expanding resilient permeable shell 140 in a smooth path radially expanded from a longitudinal axis of the permeable shell between the proximal end and distal end. The expanded state may be in the form of a torus or pumpkin, with an inverted, open distal end 150 and an inner compressible structure 148 having a lumen that extends down a longitudinal axis of the device. In some embodiments, the inner compressible structure 148 may extend down a central axis of the expanded state of the implant. The woven structure of the filaments forming the permeable shell includes a plurality of openings in each permeable shell 140 formed between the woven filaments. The braided mesh 48 of each permeable shell 140 defines an interior cavity.

The inner compressible structure 148 may have a lumen that communicates with the open distal end 150 of the permeable shell 140. The lumen of the inner compressible structure 148 may have a portion with a constant diameter. The diameter of the lumen may be between about 0.01 inches and about 0.015 inches.

The inner compressible structure 148 may be surrounded by, or structurally reinforced by, an outer constraint to provide stiffness to a proximal region of the device 110. The outer constraint may have a variable stiffness along its longitudinal axis or length. The outer constraint may have at least a proximal region and a distal region. The proximal region may be stiffer than the distal region. The outer constraint may dampen hemodynamic forces at the proximal end of the device (often visible under imaging during a contrast injection of the implantation procedure). The dampening of hemodynamic forces at the proximal end of the device may absorb the pressure changes to provide for a more securely-implanted device, reduce device migration, improve flow diversion and/or improve the rate of aneurysm size reduction over time. In some embodiments, the outer constraint may surround a portion of the inner compressible structure 148 having the constant diameter.

In some embodiments, as seen in FIGS. 1A-1B, the outer constraint may be a coil 154 surrounding at least part of the length of the inner compressible structure 148. The coil 154 may be formed by wrapping a filament or wire around at least a portion of the inner compressible structure 148. The coil 154 may have a proximal portion 252 located closer to the proximal end 142 and the proximal hub 70 of the permeable shell, and a distal portion 254 located closer to the distal end 150 of the permeable shell 140. The pitch of the coil is the axial length of one helix. As seen in FIG. 1A, the proximal portion of the coil 252 may have a different pitch (p₁) than the pitch of the distal portion of the coil (p₂). The proximal portion of the coil 252 may have a different spring constant than the spring constant of the distal portion of the coil. The winding angle or braid angle of the coil is the angle formed between the braid wire or filament with the central longitudinal axis of the helix or coil (axis 1). The proximal portion of the coil 252 may have a different winding angle (wa₁) than the winding angle of the distal portion of the coil (wa₂).

According to spring theory, the spring constant k for the coil winding is given as:

$$k = \frac{{d}^{4} G}{8 {nD}^{3}} \qquad ( 1 )$$

k=the spring constant
d=spring or filar wire diameter
G=shear modulus of the implant material
N=number of turns per unit length
D=primary wind diameter

The spring constant k determines the stiffness of the spring during deliverability. For the closely wound helical configurations in the proximal region 252 of the coil, the spring constant k is lower indicating a softer profile during implant delivery. For open gap helical configurations in the distal region 254 of the coil, the spring constant k is higher, which indicates a slight increase in stiffness profile during delivery. The stiffer central axial section of the implant is complemented by the softer torus like braid and the lack of marker band on the distal end of the implant 110. Tighter coil pitch helps reduce friction during implant delivery but at the same time demonstrates higher compressive modulus, i.e., resistance to compressive loading forces from proximal end. In contrast, the softer central axial section of the implant at proximal end is complemented by increased stiffness from the marker band creating a balanced configuration.

The coil wire diameter can range from 0.0015 inch to 0.003 inch. The pitch of the proximal portion 252 of the coil may be about 0.002 inch, alternatively about 0.01 inch, alternatively about 0.03 inch, alternatively between about 0.001 inch and 0.004 inch, alternatively between about 0.001 inch and 0.003 inch, alternatively between about 0.015 inch and 0.025 inch, alternatively between about 0.001 inch and 0.03 inch, alternatively between about 0.01 inch and 0.04 inch, alternatively between about 0.01 inch and 0.03 inch, alternatively between about 0.015 inch and 0.035 inch. The spring constant of the proximal portion 252 of the coil may be between about 0.003 to about 0.02, alternatively between about 0.004 to about 0.015, alternatively between about 0.005 to about 0.015, alternatively between about 0.005 to about 0.013, alternatively between about 0.005 to about 0.012, alternatively between about 0.006 to about 0.015, alternatively between about 0.007 to about 0.015, alternatively about 0.0060, alternatively about 0.0061, alternatively about 0.0055, alternatively about 0.005, alternatively about 0.004, alternatively about 0.003, alternatively about 0.002, alternatively about 0.015, alternatively about 0.013, alternatively about 0.011, alternatively about 0.01. The pitch may be smaller when a smaller diameter wire is used. For example, a pitch of 0.002 inch may be used with a wire having a diameter of 0.0015 inch. For implants that use a larger diameter wire, a pitch between about 0.01 to about 0.03 inch may be used to constrain the inner compressible structure 148, as well as provide axial resistance. The winding angle of the proximal portion 252 may be between about 70 and about 100 degrees, alternatively between about 75 and about 95 degrees, alternatively between about 80 and about 90 degrees. The proximal portion 252 of the coil may have a length of between about 5 mm to about 20 mm, alternatively between about 5 mm to about 18 mm, alternatively between about 7 mm to about 18 mm, alternatively between about 10 mm to about 18 mm, alternatively between about 10 mm to about 20 mm, alternatively about alternatively about 20 mm, alternatively about 18 mm, alternatively about 15 mm, alternatively about 12 mm, alternatively about 10 mm. alternatively about 7 mm, alternatively about 5 mm. The proximal portion 252 of the coil may have a length less than about 50%, alternatively less than about 40%, alternatively less than about 30%, alternatively less than about 25%, alternatively less than about 20% of the total length of the permeable shell. The proximal portion 252 of the coil may have a length less than about 50%, alternatively less than about 40%, alternatively less than about 30%, alternatively less than about 25%, alternatively less than about 20% of the total length of the coil.

The pitch of the distal portion 254 of the coil may be between about 0.0075 inch to about 0.04 inch, alternatively between about 0.0075 inch to about 0.03 inch, alternatively about 0.0075 inch, alternatively about 0.009 inch, alternatively about 0.01 inch, alternatively about 0.015 inch, alternatively about 0.02 inch, alternatively about 0.03 inch. The spring constant of the distal portion 254 of the coil may be between about 0.020 to about 0.080, alternatively between about 0.020 to about 0.075, alternatively between about 0.020 to about 0.070, alternatively about 0.020, alternatively about 0.023, alternatively about 0.027, alternatively about 0.030, alternatively about 0.033, alternatively about 0.036, alternatively about 0.040, alternatively about 0.043, alternatively about 0.046, alternatively about 0.050, alternatively about 0.053, alternatively about 0.057, alternatively about 0.060, alternatively about 0.063, alternatively about 0.067, alternatively about 0.070, alternatively about 0.075, alternatively about 0.080. The winding angle of the distal portion 254 may be between about 80 degrees and about 40 degrees, alternatively between about 75 degrees and about 45 degrees. The distal portion 254 of the coil may have a length of between about 5 mm to about 12 mm, alternatively between about 5 mm to about 11 mm, alternatively between about 5 mm to about 10 mm, alternatively about 12 mm, alternatively about 11.8 mm, alternatively about 11.6 mm, alternatively about 11.4 mm, alternatively about 11.2 mm, alternatively about 11 mm, alternatively about 10.8 mm, alternatively about 10.6 mm, alternatively about 10.4 mm, alternatively about 10.2 mm, alternatively about 10 mm, alternatively about 9.8 mm, alternatively about 9.6 mm, alternatively about 9.4 mm, alternatively about 9.2 mm, alternatively about 9.0 mm, alternatively about 8 mm, alternatively about 7 mm, alternatively about 6 mm, alternatively about 5.8 mm, alternatively about 5.6 mm, alternatively about 5.4 mm, alternatively about 5.2 mm, alternatively about 5 mm. The distal portion 254 of the coil may have a length less than about 50%, alternatively less than about 40%, alternatively less than about 30%, alternatively less than about 25%, alternatively less than about 20% of the total length of the permeable shell. The distal portion 254 of the coil may have a length less than about 50%, alternatively less than about 40%, alternatively less than about 30%, alternatively less than about 25%, alternatively less than about 20% of the total length of the coil.

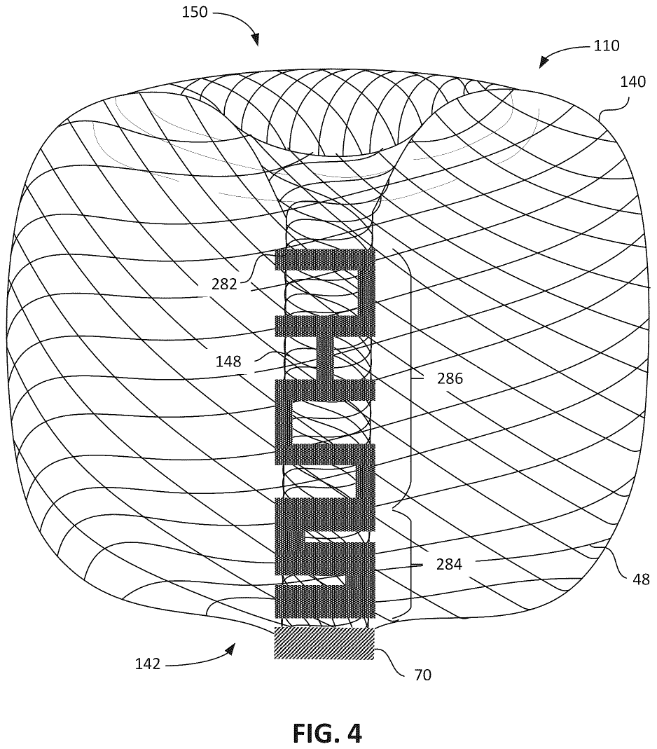

As seen in FIG. 4, in another embodiment, the outer constraint may be a laser cut or laser etched hypotube 282, where the hypotube 282 has variable stiffness along its longitudinal axis or length due to the different size of cells or openings in the proximal portion vs. the distal portion. As seen in FIG. 4, the hypotube 282 may have a proximal portion 284 having cells with an area of between about 0.05 mm² and about 0.3 mm², alternatively between about 0.059 mm² and about 0.27 mm², alternatively between about 0.1 mm² and about 0.26 mm², alternatively between about 0.1 mm² and about 0.3 mm², alternatively between about 0.1 mm² and about 0.4 mm², alternatively between about 0.1 mm² and about 0.5 mm², alternatively between about 0.1 mm² and about 0.6 mm². The distal portion 286 may have cells with an area of between about 0.10 mm² and about 1.0 mm², alternatively between about 0.15 mm² and about 0.9 mm², alternatively between about 0.17 mm² and about 0.88 mm², alternatively between about 0.28 mm² and about 0.58 mm². The hypotube thickness may vary between 0.05 mm to 0.1 mm. The hypotube strut width may vary between about 0.1 mm to about 0.2 mm.

The expanded state of the permeable shell 140 may have a maximum diameter of between about 3 mm and about 12 mm, alternatively between about 3 mm and about 10 mm, alternatively about 4 mm, alternatively about 5 mm, alternatively about 6 mm, alternatively about 7 mm, alternatively about 8 mm, alternatively about 9 mm, alternatively about 10 mm, alternatively about 11 mm. The expanded state of the permeable shell 140 can have a height or length of about 2.6 mm, about 3 mm, about 3.6 mm, about 4 mm, about 4.6 mm, about 5 mm, about 5.6 mm, about 6 mm, about 6.6 mm, about 7 mm, about 7.6 mm, about 8 mm, about 8.6 mm, about 9 mm, about 9.6 mm, or about 10 mm.

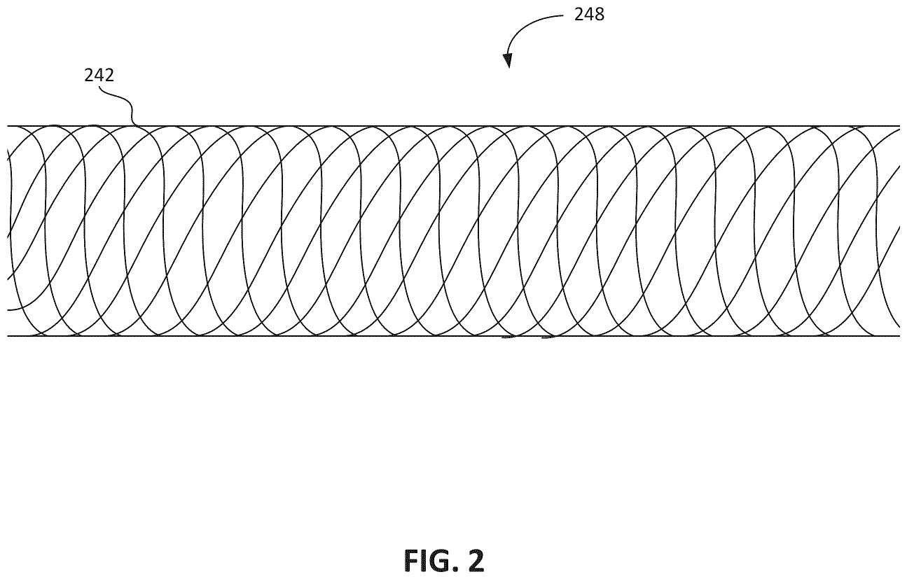

The permeable shell 140 of the implant may be made from a braided tubular mesh 248, as seen in FIG. 2. The plurality of filaments that make up the mesh or braided portion 248 may be made from nitinol, stainless steel, drawn filled tubing (e.g., platinum or tantalum core with a nitinol jacket), platinum, platinum alloys such as platinum/tungsten, or a mixture thereof. The plurality of filaments may have a diameter ranging from about 0.00075 to about 0.003 inches and may be braided on stainless steel mandrel with diameter ranging from about 3 mm to about 12 mm based on the final width of the implant design. The braided mandrel can be subsequently heat set to impart shape memory to the wires at the given mandrel diameter. FIG. 2 illustrates a primary braid wound on a mandrel based on device width before heat setting. Details of the braiding method to form the tubular mesh of FIG. 2 may be found in U.S. Pat. Nos. 8,261,648 and 8,826,791, both of which are expressly incorporated by reference herein in their entireties for all purposes.

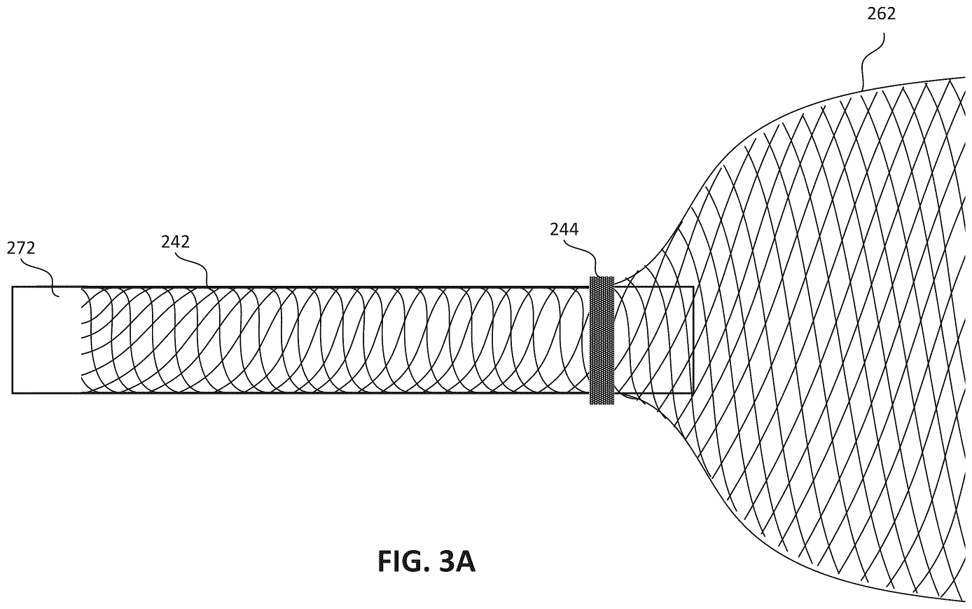

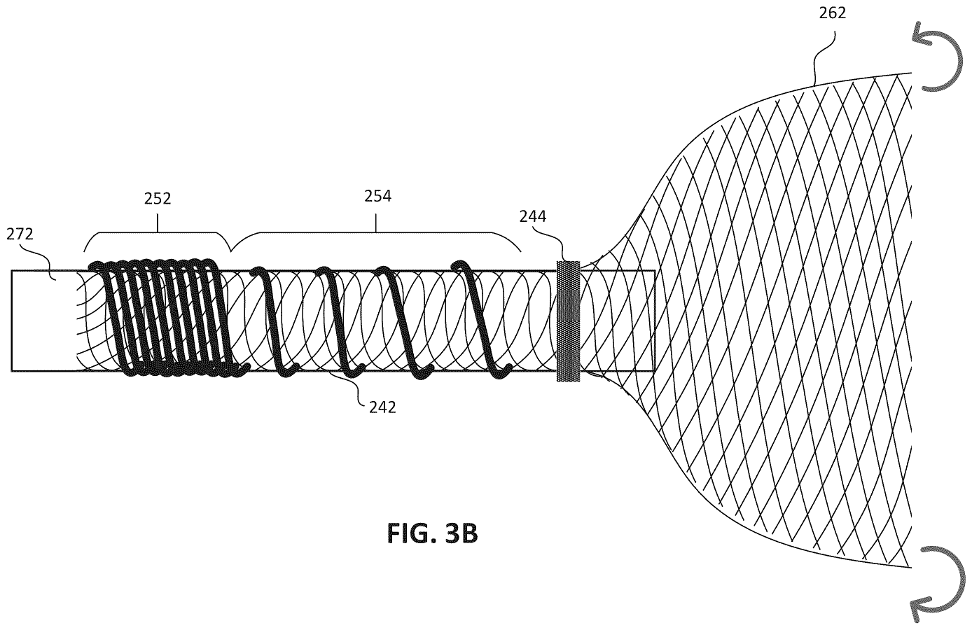

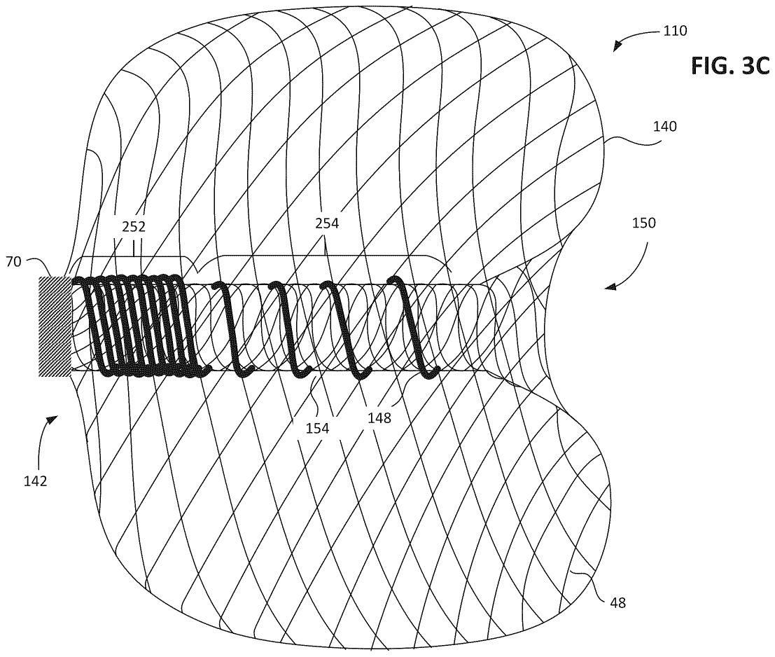

After the tubular braid 248 is removed from the mandrel, a proximal portion 242 of the tubular braid may be collapsed and loaded over a mandrel 272 having a smaller diameter than the initial mandrel used to make the tubular braid. The smaller mandrel 272 may have a diameter of between about 0.01 and about 0.015 inches, alternatively between about 0.02 inches and 0.025 inches. As seen in FIG. 3A, the interface between the funneled distal region 262 and the collapsed proximal section 242 of the braid may be constrained with a constraining feature. In some embodiments, the constraining feature may be a ring fixture 244. A similar constraining feature or mechanism may be used at the proximal end of the tubular mesh 248 as well. The tubular braid 248 may undergo a second round of heat setting to impart the collapsed structure shape memory as illustrated in the top section of FIG. 3A. The proximal constraining fixture (not shown) may then be removed, and the braided mandrel may be coil winded.

For the coil embodiment, as seen in FIG. 3B, a wire or filament may then be wound around the proximal portion 242 that was heat-set to the smaller diameter. The wire or filament may be a Platinum or Tungsten wire having an outer diameter of between about 0.0015 and about 0.003 inches. For a proximal region 252 of the proximal portion 242, the wire may be wound in a coil in a close gap configuration with the tightest possible pitch and the distal region 254, which may be about ½ to about ⅓ of the length of the proximal portion 242 or the length of the coil may be wound with a more open gap configuration.

As seen in FIG. 3B, the proximal region 252 may have a length of between about ⅓ to about ½ of the length of the proximal portion that was heat-set to the smaller diameter and may be located at a proximal end of the proximal portion 242. The winding angle of the proximal portion may be between about 70 degrees and about 95 degrees, alternatively between about 80 degrees and about 90 degrees. The pitch of the proximal region 252 may be between about 0.01 to about 0.03 inch. The proximal region 252 of the coil winding has a smaller pitch or a smaller minimal gap between revolutions than the distal region 254.

The distal region 254 of the proximal portion 242 may have a length of between about ⅓ to about ½ of the length of the proximal portion 242 that was heat-set to the smaller diameter and may be located at a distal end of the proximal portion 242. The winding angle of the distal portion may be between about 30 degrees and about 80 degrees, alternatively between about 45 degrees and about 75 degrees. The pitch of the distal region 254 may be at least about 8 times, alternatively at least about 10 times, alternatively at least about 15 times the diameter of the winding wire. The pitch of the distal region 254 may be between about 0.0075 inch and about 0.03 inch.

The ends of the coil winding wire may be welded to the collapsed braid structure to ensure the proximal portion 242 is constrained. After the wire is wound around the proximal portion 242 in the proper pitch, the mesh braid 248 may be heat set to shape set the spring mechanism in the funnel configuration. The mandrel may be removed after the shape setting is completed.

For the hypotube embodiment, as seen in FIG. 4, the laser cut or laser etched hypotube may be advanced over the proximal portion 242 that was heat-set to the smaller diameter. The hypotube may be slid over a crimped mesh braid.

After the mandrel 272 is removed, the open distal end of the funneled distal region 262 may be looped toward the proximal end such that the distal end is inverted and the formerly inner surface of the tubular mesh in the distal region 262 becomes the outer surface of the final expanded implant. Both ends of the filaments making up the tubular mesh may be gathered at the proximal end of the implant. To impart the final implant shape, a torus or pumpkin-like fixture may be used. The distal portion 262 of the mesh may be inverted and wound around the torus or pumpkin-like fixture, temporarily constrained at the proximal end of the implant and heat set. The torus or pumpkin-like fixture may then be removed. After the fixture is removed, both ends of the mesh and the coil wound around the inner compressible structure may be constrained under a marker band and laser welded. The outer constraint, e.g., coil winding or laser-cut hypotube, around the center of the collapsed braid forming the inner compressible structure may serve as a shock absorber or dampener to dampen hemodynamic forces at the proximal end. For the device with the coil, the tighter pitch at the proximal region 252 minimizes compression of the proximal end of the implant. The more open gap winding at the distal region of the coil allows the distal end of the implant to be softer.

The mesh or braided portion 48 may be made from a plurality of filaments in a woven structure that are secured relative to each other at the proximal end, e.g., in proximal marker band 70. The plurality of filaments that make up the mesh or braided portion 48 may be made from nitinol, stainless steel, drawn filled tubing (e.g., platinum or tantalum core with a nitinol jacket), platinum, platinum alloys such as platinum/tungsten, or a mixture thereof. A distal end of the mesh or braided portion 48 may be secured relative to each other at the distal end, e.g., in distal marker band 74. A proximal end of the mesh or braided portion 48 may be secured relative to each other at the proximal end, e.g., in proximal marker band 70. The wires may have a diameter of about 0.00075 inches to about 0.003 inches, alternatively about 0.001 inches to about 0.003 inches, alternatively about 0.0015 inches to about 0.0025 inches. Suitable materials and sizes of wires for constructing mesh implants are described in US 2017/0095254, US 2016/0249934, US 2016/0367260, US 2016/0249937, and US 2018/0000489, all of which are hereby expressly incorporated by reference in their entirety for all purposes.

Delivery and deployment of device embodiment 110 discussed herein may be carried out by first compressing the device 110 to a radially constrained and longitudinally flexible state. The device 110 may be attached to a pusher that can be advanced through the lumen of the microcatheter. The marker band of the device 110 may be releasably attached to the pusher. The device 110 may then be delivered to a desired treatment site, e.g., aneurysm 160, while disposed within the microcatheter, and then ejected or otherwise deployed from a distal end of the microcatheter. In other method embodiments, the microcatheter may first be navigated to a desired treatment site over a guidewire or by other suitable navigation techniques. The distal end of the microcatheter may be positioned such that a distal port of the microcatheter is directed towards or disposed within a vascular defect 160 to be treated and the guidewire withdrawn. The device 110 secured to a suitable delivery apparatus and in a radially constrained configuration, and having been inserted into a proximal portion of the inner lumen of the microcatheter, may be distally advanced to the vascular defect 160 through the inner lumen.

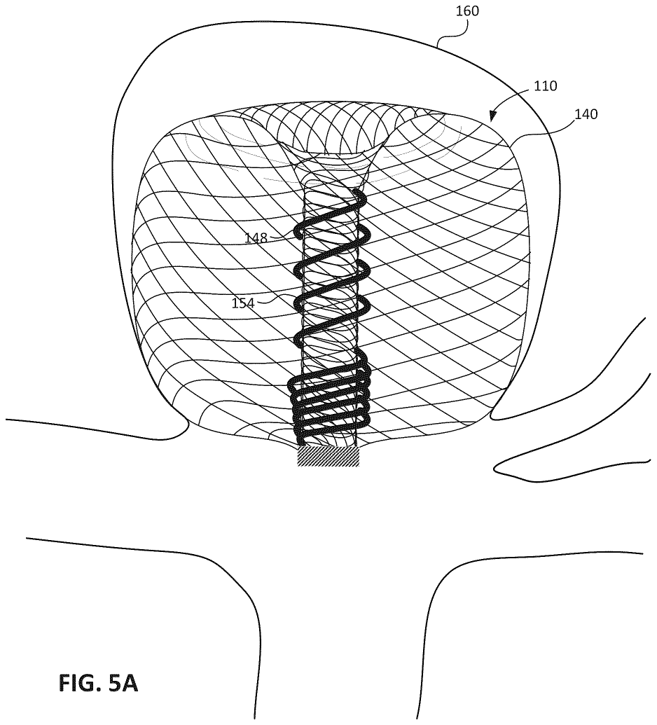

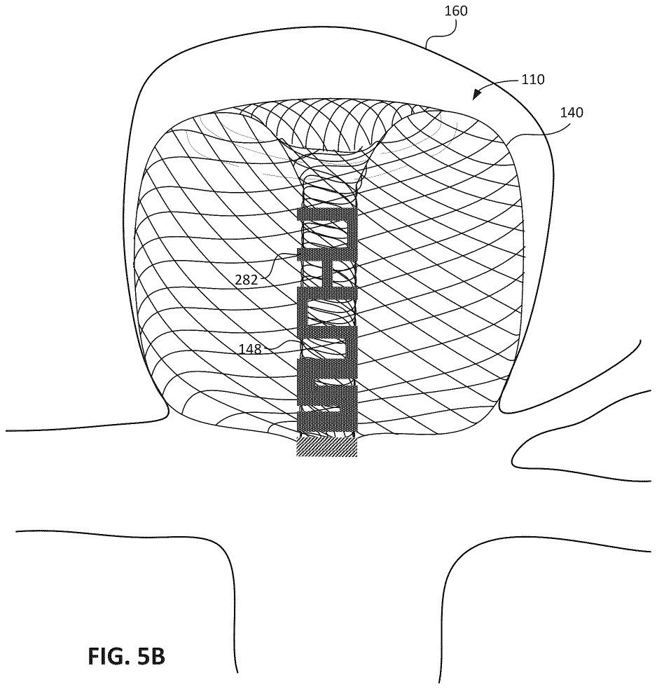

As seen in FIGS. 5A-5B, once disposed within the vascular defect 160, such as a wide-necked aneurysm, the device 110 may then be allowed to assume an expanded relaxed or partially relaxed state with the permeable shell 140 of the device spanning or partially spanning a portion of the vascular defect 160 or the entire vascular defect 160. Once the device 110 is deployed at a desired treatment site, and the permeable shell 140 has been detached from the delivery device(s), the microcatheter may then be withdrawn. The device 110 may be detached via a mechanical, chemical, or electrothermal mechanism, for example, V-Trak™ (MicroVention, Inc., Aliso Viejo, CA) and/or mechanisms as described in U.S. Pat. Nos. 8,182,506, 9,414,819, 9,242,070, 10,076,338, 8,192,480, 9,717,500, 9,968,358, 8,932,317, 9,561,125, 9,949,739, 9,867,622, 11,039,840 and PCT Patent Application Nos. PCT/US2022/0735096 and PCT/US2022/071230, all of which are hereby expressly incorporated by reference in its entireties for all purposes. The outer constraint may have at least a proximal region and a distal region. The proximal region may be stiffer than the distal region. The outer constraint may dampen hemodynamic forces at the proximal end of the device.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

Aspects of the invention are set out in the independent claims and preferred features are set out in the dependent claims. The preferred features of the dependent claims may be provided in combination in a single embodiment and preferred features of one aspect may be provided in conjunction with other aspects.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, a device for treatment of a patient's aneurysm includes: a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and an outer constraint surrounding at least a portion of the inner compressible mesh structure, the outer constraint comprising a variable stiffness along a length of the outer constraint.

In some embodiments, the outer constraint comprises a proximal portion and a distal portion, wherein the proximal portion of the outer constraint is stiffer than the distal portion of the outer constraint.

In some embodiments, the hub is radiopaque.

In some embodiments, the device further comprises a marker band surrounding the hub.

In some embodiments, the outer constraint comprises a coil formed from a wire, wherein the coil comprises a proximal portion having a first pitch and a distal portion having a second pitch, wherein the first pitch is tighter than the second pitch.

In some embodiments, the proximal portion of the coil has a first spring constant and the distal portion has a second spring constant, wherein the first spring constant is lower than the second spring constant. In some embodiments, the proximal portion of the coil has a spring constant between about 0.005 and about 0.015. In some embodiments, the distal portion of the coil has a spring constant between about 0.02 and about 0.07.

In some embodiments, the first pitch is at least two times the second pitch. In some embodiments, the first pitch is between about 0.01 and about 0.03 inch. In some embodiments, the second pitch is between about 0.007 and about 0.03 inch.

In some embodiments, the proximal portion of the coil has a winding angle of between about 80 degrees and about 90 degrees. In some embodiments, the distal portion of the coil has a winding angle of between about 45 degrees and about 75 degrees.

In some embodiments, the second pitch is at least about 10 times a diameter of the wire forming the coil.

In some embodiments, a first end of the wire is coupled to a proximal region of the inner compressible mesh structure. In some embodiments, a second end of the wire is coupled to a distal region of the inner compressible mesh structure.

In some embodiments, the distal end of the permeable shell is inverted.

In some embodiments, wherein the permeable shell has an open end and wherein the inner compressible mesh structure has a lumen that communicates with the open end of the permeable shell. In some embodiments, the lumen has a diameter of between about 0.01 and about 0.015 inches.

In some embodiments, the inner compressible mesh structure extends down a central longitudinal axis of the permeable shell.

In some embodiments, the proximal portion of the outer constraint has a length that is less than about 50% of a total length of the permeable shell.

In some embodiments, the proximal portion of the outer constraint has a length that is less than about 40% of a total length of the permeable shell.

In some embodiments, the distal portion of the outer constraint has a length that is less than 50% of a total length of the permeable shell.

In some embodiments, the distal portion of the outer constraint has a length that is less than 40% of a total length of the permeable shell.

In some embodiments, the proximal portion of the outer constraint has a length that is less than about 50% of a total length of the outer constraint.

In some embodiments, the distal portion of the outer constraint has a length that is less than 50% of a total length of the outer constraint.

In some embodiments, the outer constraint is a laser-cut hypotube comprising a proximal portion and a distal portion, wherein the proximal portion comprises openings having a first area and the distal portion comprises openings having a second area, and wherein the first area is smaller than the second area.

In many embodiments, a method for treating a cerebral aneurysm having an interior cavity and a neck includes the steps of: advancing an implant in a microcatheter to a region of interest in a cerebral artery, wherein the implant comprises: a permeable shell comprising a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure; and an outer constraint surrounding at least a portion of the inner compressible mesh structure, the outer constraint comprising a variable stiffness along a length of the outer constraint, and a hub at the proximal end of the permeable shell, wherein the first and second ends of each of the plurality of filaments are held in the marker band; deploying the implant within the cerebral aneurysm, wherein the permeable shell expands to the expanded state in the interior cavity of the aneurysm; and withdrawing the microcatheter from the region of interest after deploying the implant.

In some embodiments, the outer constraint comprises a proximal portion and a distal portion, wherein the proximal portion of the outer constraint is stiffer than the distal portion of the outer constraint.

In some embodiments, the hub is radiopaque.

In some embodiments, the device further comprises a marker band surrounding the hub.

In some embodiments, the outer constraint comprises a coil formed from a wire, wherein the coil comprises a proximal portion having a first pitch and a distal portion having a second pitch, wherein the first pitch is tighter than the second pitch.

In some embodiments, the proximal portion of the coil has a first spring constant and the distal portion has a second spring constant, wherein the first spring constant is lower than the second spring constant. In some embodiments, the proximal portion of the coil has a spring constant between about 0.005 and about 0.015. In some embodiments, the distal portion of the coil has a spring constant between about 0.02 and about 0.07.

In some embodiments, the first pitch is at least two times the second pitch.

In some embodiments, the first pitch is between about 0.01 and about 0.03 inch.

In some embodiments, the second pitch is between about 0.007 and about 0.03 inch.

In some embodiments, the proximal portion of the coil has a winding angle of between about 80 degrees and about 90 degrees.

In some embodiments, the distal portion of the coil has a winding angle of between about 45 degrees and about 75 degrees.

In some embodiments, the second pitch is at least about 10 times a diameter of the wire forming the coil.

In some embodiments, a first end of the wire is coupled to a proximal region of the inner compressible mesh structure.

In some embodiments, a second end of the wire is coupled to a distal region of the inner compressible mesh structure.

In some embodiments, the distal end of the permeable shell is inverted.

In some embodiments, the permeable shell has an open end and wherein the inner compressible mesh structure has a lumen that communicates with the open end of the permeable shell. In some embodiments, the lumen has a diameter of between about 0.01 and about 0.015 inches.

In some embodiments, the inner compressible mesh structure extends down a central longitudinal axis of the permeable shell.

In some embodiments, the proximal portion of the outer constraint has a length that is less than about 50% of a total length of the permeable shell.

In some embodiments, the proximal portion of the outer constraint has a length that is less than about 40% of a total length of the permeable shell.

In some embodiments, the distal portion of the outer constraint has a length that is less than 50% of a total length of the permeable shell.

In some embodiments, the distal portion of the outer constraint has a length that is less than 40% of a total length of the permeable shell.

In some embodiments, the proximal portion of the outer constraint has a length that is less than about 50% of a total length of the outer constraint.

In some embodiments, the distal portion of the outer constraint has a length that is less than 50% of a total length of the outer constraint.

In some embodiments, the outer constraint is a laser-cut hypotube comprising a proximal portion and a distal portion, wherein the proximal portion comprises openings having a first area and the distal portion comprises openings having a second area, and wherein the first area is smaller than the second area.

In many embodiments, a device for treatment of a patient's aneurysm includes: a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and a constraining means for located around at least a portion of the inner compressible mesh structure for dampening hemodynamic forces at the proximal end of the permeable shell.

In some embodiments, the constraining means comprises a proximal portion and a distal portion, wherein the proximal portion of the constraining means is stiffer than the distal portion of the outer constraint.

In some embodiments, the constraining means is a coil having a variable stiffness.

In some embodiments, the coil formed is from a wire, wherein the coil comprises a proximal portion having a first pitch and a distal portion having a second pitch, wherein the first pitch is tighter than the second pitch.

In some embodiments, the proximal portion of the coil has a first spring constant and the distal portion has a second spring constant, wherein the first spring constant is lower than the second spring constant.

In some embodiments, the proximal portion of the coil has a spring constant between about 0.005 and about 0.015.

In some embodiments, the distal portion of the coil has a spring constant between about 0.02 and about 0.07.

In some embodiments, the first pitch is at least two times the second pitch.

In some embodiments, the first pitch is between about 0.01 and about 0.03 inch.

In some embodiments, the second pitch is between about 0.007 and about 0.03 inch.

In some embodiments, the proximal portion of the coil has a winding angle of between about 80 degrees and about 90 degrees.

In some embodiments, the distal portion of the coil has a winding angle of between about 45 degrees and about 75 degrees.

In some embodiments, the constraining means is a hypotube having a variable stiffness.

In some embodiments, the hypotube is laser cut with a plurality of openings.

### CLAUSES

Exemplary embodiments are set out in the following numbered clauses.

Clause 1. A device for treatment of a patient's aneurysm, comprising:
a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and
an outer constraint surrounding at least a portion of the inner compressible mesh structure, the outer constraint comprising a variable stiffness along a length of the outer constraint.

Clause 2. The device of clause 1, wherein the outer constraint comprises a proximal portion and a distal portion, wherein the proximal portion of the outer constraint is stiffer than the distal portion of the outer constraint.

Clause 3. The device of clause 1, wherein the hub is radiopaque.

Clause 4. The device of clause 1, wherein the device further comprises a marker band surrounding the hub.

Clause 5. The device of clause 1, wherein the outer constraint comprises a coil formed from a wire, wherein the coil comprises a proximal portion having a first pitch and a distal portion having a second pitch, wherein the first pitch is tighter than the second pitch.

Clause 6. The device of clause 5, wherein the proximal portion of the coil has a first spring constant and the distal portion has a second spring constant, wherein the first spring constant is lower than the second spring constant.

Clause 7. The device of clause 6, wherein the proximal portion of the coil has a spring constant between about 0.005 and about 0.015.

Clause 8. The device of clause 6, wherein the distal portion of the coil has a spring constant between about 0.02 and about 0.07.

Clause 9. The device of clause 5, wherein the first pitch is at least two times the second pitch.

Clause 10. The device of clause 5, wherein the first pitch is between about 0.01 and about 0.03 inch.

Clause 11. The device of clause 5, wherein the second pitch is between about 0.007 and about 0.03 inch.

Clause 12. The device of clause 5, wherein the proximal portion of the coil has a winding angle of between about 80 degrees and about 90 degrees.

Clause 13. The device of clause 5, wherein the distal portion of the coil has a winding angle of between about 45 degrees and about 75 degrees.

Clause 14. The device of clause 5, wherein the second pitch is at least about 10 times a diameter of the wire forming the coil.

Clause 15. The device of clause 5, wherein a first end of the wire is coupled to a proximal region of the inner compressible mesh structure.

Clause 16. The device of clause 5, wherein a second end of the wire is coupled to a distal region of the inner compressible mesh structure.

Clause 17. The device of clause 1, wherein the distal end of the permeable shell is inverted.

Clause 18. The device of clause 1, wherein the permeable shell has an open end and wherein the inner compressible mesh structure has a lumen that communicates with the open end of the permeable shell.

Clause 19. The device of clause 18, the lumen has a diameter of between about 0.01 and about 0.015 inches.

Clause 20. The device of clause 1, wherein the inner compressible mesh structure extends down a central longitudinal axis of the permeable shell.

Clause 21. The device of clause 1, wherein the proximal portion of the outer constraint has a length that is less than about 50% of a total length of the permeable shell.

Clause 22. The device of clause 1, wherein the proximal portion of the outer constraint has a length that is less than about 40% of a total length of the permeable shell.

Clause 23. The device of clause 1, wherein the distal portion of the outer constraint has a length that is less than 50% of a total length of the permeable shell.

Clause 24. The device of clause 1, wherein the distal portion of the outer constraint has a length that is less than 40% of a total length of the permeable shell.

Clause 25. The device of clause 1, wherein the proximal portion of the outer constraint has a length that is less than about 50% of a total length of the outer constraint.

Clause 26. The device of clause 1, wherein the distal portion of the outer constraint has a length that is less than 50% of a total length of the outer constraint.

Clause 27. The device of clause 1, wherein the outer constraint is a laser-cut hypotube comprising a proximal portion and a distal portion, wherein the proximal portion comprises openings having a first area and the distal portion comprises openings having a second area, and wherein the first area is smaller than the second area.

Clause 28. A method for treating a cerebral aneurysm having an interior cavity and a neck, comprising the steps of:
advancing an implant in a microcatheter to a region of interest in a cerebral artery, wherein the implant comprises:
   a permeable shell comprising a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure; and
   an outer constraint surrounding at least a portion of the inner compressible mesh structure, the outer constraint comprising a variable stiffness along a length of the outer constraint, and a hub at the proximal end of the permeable shell, wherein the first and second ends of each of the plurality of filaments are held in the marker band;
deploying the implant within the cerebral aneurysm, wherein the permeable shell expands to the expanded state in the interior cavity of the aneurysm; and
withdrawing the microcatheter from the region of interest after deploying the implant.

Clause 29. The method of clause 28, wherein the outer constraint comprises a proximal portion and a distal portion, wherein the proximal portion of the outer constraint is stiffer than the distal portion of the outer constraint.

Clause 30. The method of clause 28, wherein the hub is radiopaque.

Clause 31. The method of clause 28, wherein the device further comprises a marker band surrounding the hub.

Clause 32. The method of clause 28, wherein the outer constraint comprises a coil formed from a wire, wherein the coil comprises a proximal portion having a first pitch and a distal portion having a second pitch, wherein the first pitch is tighter than the second pitch.

Clause 33. The method of clause 32, wherein the proximal portion of the coil has a first spring constant and the distal portion has a second spring constant, wherein the first spring constant is lower than the second spring constant.

Clause 34. The method of clause 33, wherein the proximal portion of the coil has a spring constant between about 0.005 and about 0.015.

Clause 35. The method of clause 33, wherein the distal portion of the coil has a spring constant between about 0.02 and about 0.07.

Clause 36. The method of clause 32, wherein the first pitch is at least two times the second pitch.

Clause 37. The method of clause 32, wherein the first pitch is between about 0.01 and about 0.03 inch.

Clause 38. The method of clause 32, wherein the second pitch is between about 0.007 and about 0.03 inch.

Clause 39. The method of clause 32, wherein the proximal portion of the coil has a winding angle of between about 80 degrees and about 90 degrees.

Clause 40. The method of clause 32, wherein the distal portion of the coil has a winding angle of between about 45 degrees and about 75 degrees.

Clause 41. The method of clause 32, wherein the second pitch is at least about 10 times a diameter of the wire forming the coil.

Clause 42. The method of clause 32, wherein a first end of the wire is coupled to a proximal region of the inner compressible mesh structure.

Clause 43. The method of clause 32, wherein a second end of the wire is coupled to a distal region of the inner compressible mesh structure.

Clause 44. The method of clause 28, wherein the distal end of the permeable shell is inverted.

Clause 45. The method of clause 28, wherein the permeable shell has an open end and wherein the inner compressible mesh structure has a lumen that communicates with the open end of the permeable shell.

Clause 46. The method of clause 45, the lumen has a diameter of between about 0.01 and about 0.015 inches.

Clause 47. The method of clause 28, wherein the inner compressible mesh structure extends down a central longitudinal axis of the permeable shell.

Clause 48. The method of clause 28, wherein the proximal portion of the outer constraint has a length that is less than about 50% of a total length of the permeable shell.

Clause 49. The method of clause 28, wherein the proximal portion of the outer constraint has a length that is less than about 40% of a total length of the permeable shell.

Clause 50. The method of clause 28, wherein the distal portion of the outer constraint has a length that is less than 50% of a total length of the permeable shell.

Clause 51. The method of clause 28, wherein the distal portion of the outer constraint has a length that is less than 40% of a total length of the permeable shell.

Clause 52. The method of clause 28, wherein the proximal portion of the outer constraint has a length that is less than about 50% of a total length of the outer constraint.

Clause 53. The method of clause 28, wherein the distal portion of the outer constraint has a length that is less than 50% of a total length of the outer constraint.

Clause 54. The method of clause 28, wherein the outer constraint is a laser-cut hypotube comprising a proximal portion and a distal portion, wherein the proximal portion comprises openings having a first area and the distal portion comprises openings having a second area, and wherein the first area is smaller than the second area.

Clause 55. A device for treatment of a patient's aneurysm, comprising:
a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and
a constraining means for located around at least a portion of the inner compressible mesh structure for dampening hemodynamic forces at the proximal end of the permeable shell.

Clause 56. The device of clause 55, wherein the constraining means comprises a proximal portion and a distal portion, wherein the proximal portion of the constraining means is stiffer than the distal portion of the outer constraint.

Clause 57. The device of clause 55, wherein the constraining means is a coil having a variable stiffness.

Clause 58. The device of clause 57, wherein the coil formed is from a wire, wherein the coil comprises a proximal portion having a first pitch and a distal portion having a second pitch, wherein the first pitch is tighter than the second pitch.

Clause 59. The device of clause 58, wherein the proximal portion of the coil has a first spring constant and the distal portion has a second spring constant, wherein the first spring constant is lower than the second spring constant.

Clause 60. The device of clause 58, wherein the proximal portion of the coil has a spring constant between about 0.005 and about 0.015.

Clause 61. The device of clause 58, wherein the distal portion of the coil has a spring constant between about 0.02 and about 0.07.

Clause 62. The device of clause 58, wherein the first pitch is at least two times the second pitch.

Clause 63. The device of clause 58, wherein the first pitch is between about 0.01 and about 0.03 inch.

Clause 64. The device of clause 58, wherein the second pitch is between about 0.007 and about 0.03 inch.

Clause 65. The device of clause 58, wherein the proximal portion of the coil has a winding angle of between about 80 degrees and about 90 degrees.

Clause 66. The device of clause 58, wherein the distal portion of the coil has a winding angle of between about 45 degrees and about 75 degrees.

Clause 67. The device of clause 57, wherein the constraining means is a hypotube having a variable stiffness.

Clause 68. The device of clause 67, wherein the hypotube is laser cut with a plurality of openings.

1. A device for treatment of a patient's aneurysm, comprising:
a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and
an outer constraint surrounding at least a portion of the inner compressible mesh structure, the outer constraint comprising a variable stiffness along a length of the outer constraint.

2. The device of claim 1, wherein the outer constraint comprises a proximal portion and a distal portion, wherein the proximal portion of the outer constraint is stiffer than the distal portion of the outer constraint.

3.-4. (canceled)

5. The device of claim 1, wherein the outer constraint comprises a coil formed from a wire, wherein the coil comprises a proximal portion having a first pitch and a distal portion having a second pitch, wherein the first pitch is tighter than the second pitch.

6. The device of claim 5, wherein the proximal portion of the coil has a first spring constant and the distal portion has a second spring constant, wherein the first spring constant is lower than the second spring constant.

7. The device of claim 6, wherein the proximal portion of the coil has a spring constant between about 0.005 and about 0.015.

8. (canceled)

9. The device of claim 5, wherein the first pitch is at least two times the second pitch.

10. The device of claim 5, wherein the first pitch is between about 0.01 and about 0.03 inch.

11. (canceled)

12. The device of claim 5, wherein the proximal portion of the coil has a winding angle of between about 80 degrees and about 90 degrees.

13. (canceled)

14. The device of claim 5, wherein the second pitch is at least about 10 times a diameter of the wire forming the coil.

15. (canceled)

16. The device of claim 5, wherein a second end of the wire is coupled to a distal region of the inner compressible mesh structure.

17. The device of claim 1, wherein the distal end of the permeable shell is inverted.

18. The device of claim 1, wherein the permeable shell has an open end and wherein the inner compressible mesh structure has a lumen that communicates with the open end of the permeable shell.

19. The device of claim 18, the lumen has a diameter of between about 0.01 and about 0.015 inches.

20. The device of claim 1, wherein the inner compressible mesh structure extends down a central longitudinal axis of the permeable shell.

21. The device of claim 1, wherein the proximal portion of the outer constraint has a length that is less than about 50% of a total length of the permeable shell.

22. The device of claim 1, wherein the proximal portion of the outer constraint has a length that is less than about 40% of a total length of the permeable shell.

23. The device of claim 1, wherein the distal portion of the outer constraint has a length that is less than 50% of a total length of the permeable shell.

24.-26. (canceled)

27. The device of claim 1, wherein the outer constraint is a laser-cut hypotube comprising a proximal portion and a distal portion, wherein the proximal portion comprises openings having a first area and the distal portion comprises openings having a second area, and wherein the first area is smaller than the second area.

28. A method for treating a cerebral aneurysm having an interior cavity and a neck, comprising the steps of:
advancing an implant in a microcatheter to a region of interest in a cerebral artery, wherein the implant comprises:
a permeable shell comprising a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure; and
an outer constraint surrounding at least a portion of the inner compressible mesh structure, the outer constraint comprising a variable stiffness along a length of the outer constraint, and a hub at the proximal end of the permeable shell, wherein the first and second ends of each of the plurality of filaments are held in the marker band;
deploying the implant within the cerebral aneurysm, wherein the permeable shell expands to the expanded state in the interior cavity of the aneurysm; and
withdrawing the microcatheter from the region of interest after deploying the implant.

29.-54. (canceled)

55. A device for treatment of a patient's aneurysm, comprising:
a permeable shell including a proximal end, a distal end, a radially constrained elongated state configured for delivery within a catheter lumen, an expanded state, and a plurality of filaments that are woven together to form a mesh, wherein each of the plurality of filaments has a first end and a second end, wherein each of the plurality of filaments starts at the proximal end of the permeable shell, extends to the distal end of the permeable shell, and extends back to the proximal end of the permeable shell forming an inner compressible mesh structure, and wherein the first and second ends of each of the plurality of filaments are gathered in a hub at the first end of the first permeable shell; and
a constraining means for located around at least a portion of the inner compressible mesh structure for dampening hemodynamic forces at the proximal end of the permeable shell.

56.-68. (canceled)
